(19) United States
(12) Patent Application Publication
(10) Pub. No.: US 2018/0291077 A1
(43) Pub. Date: Oct. 11, 2018

(54) METHOD OF INSULIN PRODUCTION

(71) Applicant: HANMI PHARM. CO., LTD, Hwaseong-si, Gyeonggi-do (KR)

(72) Inventors: Seong Ho CHOI, Hwaseong-si (KR); Dae Jin KIM, Hwaseong-si (KR); Jin Young KIM, Hwaseong-si (KR); Sang Yun KIM, Hwaseong-si (KR); In Young CHOI, Hwaseong-si (KR); Se Chang KWON, Hwaseong-si (KR)

(73) Assignee: HANMI PHARM. CO., LTD, Hwaseong-si, Gyeonggi-do (KR)

(21) Appl. No.: 15/762,613

(22) PCT Filed: Sep. 23, 2016
(86) PCT No.: PCT/KR2016/010713

§ 371 Date: Mar. 23, 2018

(30) Foreign Application Priority Data
Sep. 24, 2015 (KR) 10-2015-0135872

(51) Int. Cl. C07K 14/62; B01D 15/36; B01D 15/32; C07K 1/18; C07K 1/20

(52) U.S. Cl. CPC C07K 14/62; B01D 15/362; C07K 1/20; C07K 1/18; B01D 15/325

(57) ABSTRACT

The present invention relates to a method of preparing insulin from proinsulin comprising converting high-concentration proinsulin into insulin by enzymatic cleavage, a method of purifying insulin, and insulin prepared therefrom.

### TECHNICAL FIELD

The present invention relates to a method of preparing insulin from proinsulin comprising converting high-concentration proinsulin into insulin by enzymatic cleavage, a method of purifying insulin, and the insulin prepared using the same methods.

### BACKGROUND ART

The method of preparing recombinant insulin has been continuously developed from the method of preparing semi-synthetic insulin to a two-chain method and to a method of preparing insulin from proinsulin.

Among the methods of preparing insulin from proinsulin, excluding the two-chain method and the semi-synthetic method, the process of converting proinsulin into insulin using trypsin and carboxypeptidase B have been used for many years [see Kemmler, W., Clark, j., Steiner, D. F., Fed. Proc. 30 (1971) 1210; Kemmler, W., Peterson, J. D., Steiner, D. F., J. Biol. Chem., 246 (1971) 6788-6791]. However, when insulin is prepared by this method, impurities, which are difficult to remove by the general purification method such as using a column, etc., in particular, a type of human insulin wherein the last amino acid in the B-chain, threonine, is deleted [Des-Thr(B30)-insulin], are mostly formed in large quantities (4% to 10%), compared with other impurities generated during insulin production, although the content of the impurities varies according to the conditions.

When the semi-synthetic method is used, the C-peptide is modified to a form different from that of the wild-type so that it can be removed by a simple treatment with trypsin in a given proinsulin analog and the methods generates a form wherein the last amino acid in the B-chain, threonine, is deleted. Then, L-threonine t-butyl ester is attached to the last amino acid of the B-chain of the thus-prepared insulin via synthesis, and the thus-prepared insulin-ester and the human insulin form, wherein the last amino acid in the B-chain, threonine, is deleted [Des-Thr(B30)-insulin], are isolated. In contrast, when human proinsulin is used as an intermediate, Des-Thr(B30)-insulin is generated in a large amount during the process of enzymatic conversion, and thus various attempts have been made to inhibit its generation.

For example, U.S. Pat. No. 5,457,066 discloses that the amount of Des-Thr(B30)-insulin production was reduced by introducing a second metal ion in the enzyme conversion process.

Additionally, according to Son Y J et al. (Biotechnol Prog. 2009 July-August; 25(4):1064-70) and US Patent Application Publication No. 2012-0214964, the amount of Des-Thr(B30)-insulin production was reduced by performing an enzyme conversion process, after performing a reaction to block the B29 lysine site near the B30 threonine via ‘citraconylation’.

However, these methods may give rise to potential problems due to additives during the purification process performed following the enzyme conversion process. Additionally, these methods may also have a problem in that a step of adding an additive to inhibit the generation of the Des-Thr(B30)-insulin and/or unblocking the additive is further required in the above process, thereby increasing the procedural complexity and leading to an increase in the production cost.

### DISCLOSURE

### Technical Problem

The present inventors have endeavored to develop a method to minimize the production of impurities in the method of preparing insulin using proinsulin as an intermediate, and as a result, have developed a method of performing an enzyme conversion process on a high-concentration proinsulin sample. Accordingly, the production of Des-Thr(B30)-insulin can be effectively reduced by the method developed in the present invention.

### Technical Solution

An object of the present invention is to provide a method for preparing insulin from proinsulin, which comprises converting high-concentration proinsulin into insulin by enzymatic cleavage.

Another object of the present invention is to provide a method for purifying insulin comprising (a) preparing an insulin-containing sample by converting high-concentration proinsulin into insulin by enzymatic cleavage; and (b) subjecting the sample to a purification process.

A further object of the present invention is to provide insulin prepared by the above method.

### Advantageous Effects

The method of the present invention can prepare an insulin sample, where impurities are effectively controlled, and thus can significantly improve insulin purification efficiency. Accordingly, the method of the present invention can be applied to large-scale production of insulin, and is thus capable of reducing cost for removing impurities.

### DESCRIPTION OF DRAWINGS

FIG. 1 shows a graph illustrating the analysis result of an impurities reduction effect according to proinsulin concentration, when treated with an enzyme.

FIG. 2 shows a graph illustrating the analysis result of an impurities reduction effect according to a reaction temperature condition, when treated with an enzyme.

FIG. 3 shows a graph illustrating the analysis result of an impurities reduction effect according to pH, when treated with an enzyme.

FIG. 4 shows a graph illustrating the result of analysis by reversed phase chromatography of an insulin analog sample containing an excess amount of Des-Thr(B30)-insulin analog.

FIGS. 5A to 5C show graphs illustrating the analysis results of the purity of insulin analogs purified by high-pressure chromatography (HPLC); i.e., FIG. 5a by C18 RP-HPLC, FIG. 5b by C4 RP-HPLC, and FIG. 5c by SEC-HPLC.

### BEST MODE

In an aspect to achieve the above objects, the present invention provides a method for preparing insulin from proinsulin comprising converting proinsulin at a concentration of 50 mg/mL or higher into insulin by enzymatic cleavage.

In an exemplary embodiment, the enzyme is trypsin, carboxypeptidase B, or a combination thereof.

In another exemplary embodiment, the concentration of the proinsulin is in the range from 50 mg/mL to 300 mg/mL.

In still another exemplary embodiment, the concentration of the proinsulin is in the range from 100 mg/mL to 300 mg/mL.

In still another exemplary embodiment, the concentration of the proinsulin is in the range from 200 mg/mL to 300 mg/mL.

In still another exemplary embodiment, the percentage of trypsin relative to proinsulin is in the range from 1/7,500 to 1/40,000 (weight/weight).

In still another exemplary embodiment, the percentage of trypsin relative to proinsulin is in the range from 1/15,000 to 1/40,000 (weight/weight).

In still another exemplary embodiment, the percentage of trypsin relative to proinsulin is in the range from 1/20,000 to 1/40,000 (weight/weight).

In still another exemplary embodiment, the percentage of trypsin relative to proinsulin is in the range from 1/30,000 to 1/40,000 (weight/weight).

In still another exemplary embodiment, the percentage of carboxypeptidase B relative to proinsulin is in the range from 1/600 to 1/20,000 (weight/weight).

In still another exemplary embodiment, the percentage of carboxypeptidase B relative to proinsulin is in the range from 1/600 to 1/15,000 (weight/weight).

In still another exemplary embodiment, the pH in the enzyme reaction is in the range from 6.5 to 9.0.

In still another exemplary embodiment, the pH in the enzyme reaction is in the range from 7.0 to 8.5.

In still another exemplary embodiment, the temperature in the enzyme reaction is in the range from 4.0° C. to 25.0° C.

In still another exemplary embodiment, the reaction time in the enzyme reaction is in the range from 4.0 hours to 55 hours.

In still another exemplary embodiment, the buffer in the enzyme reaction is in the range from 1 mM to 100 mM Tris-HCl.

In still another exemplary embodiment, the buffer in the enzyme reaction may not comprise the metal ion.

In still another exemplary embodiment, the proinsulin or insulin is in an analog type.

In still another exemplary embodiment, the method further comprises purifying insulin by subjecting a sample containing the insulin converted from proinsulin to chromatography.

In still another exemplary embodiment, the chromatography is cation exchange chromatography or reversed phase chromatography.

In still another exemplary embodiment, the method further comprises performing reversed phase chromatography or anion exchange chromatography.

In still another exemplary embodiment, the method further comprises performing reversed phase chromatography, after purifying insulin by subjecting a sample containing the insulin converted from proinsulin to cation exchange chromatography.

In still another exemplary embodiment, the proinsulin is partially purified by a cation exchange column or a reversed column.

In still another exemplary embodiment, the sample containing insulin prepared by the method contains Des-Thr(B30)-insulin impurities in the amount of less than 5%.

In another aspect to achieve the objects, the present invention provides a method for purifying insulin comprising preparing an insulin-containing sample by converting high-concentration proinsulin into insulin by enzymatic cleavage; and subjecting the thus-prepared sample to a purification process.

In an exemplary embodiment, the chromatography is cation exchange chromatography or reversed phase chromatography.

In another exemplary embodiment, the method comprises purifying insulin by subjecting a sample containing the insulin, which was converted from proinsulin, to cation exchange chromatography followed by performing reversed phase chromatography.

In a further aspect to achieve the objects, the present invention provides insulin prepared by the above method.

### MODE FOR INVENTION

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

In an aspect to achieve the above objects, the present invention provides a method of preparing insulin from proinsulin comprising converting high-concentration proinsulin into insulin by enzymatic cleavage.

In the method of the present invention, the proinsulin may be used at high concentration.

Specifically, proinsulin at a concentration of 50 mg/mL or higher may be used in enzymatic conversion. More specifically, in the above method, the concentration of the proinsulin may be used in the range from 50 mg/mL to 300 mg/mL, even more specifically from 100 mg/mL to 300 mg/mL, and most specifically from 200 mg/mL to 300 mg/mL, but it is not limited thereto.

In the present invention, the conversion of proinsulin into insulin by enzymatic cleavage is also called enzymatic conversion.

As used herein, the term “enzymatic conversion” refers to a conversion of proinsulin containing a C-peptide between the A-chain and the B-chain into insulin using an enzyme.

In the present invention, the enzymatic conversion may be performed using any one selected from trypsin, carboxypeptidase B, and a combination thereof.

In the present invention, the percentage of trypsin relative to proinsulin may be used in the range from 1/7,500 to 1/40,000 (weight/weight), specifically from 1/15,000 to 1/40,000 (weight/weight), more specifically from 1/20,000 to 1/40,000 (weight/weight), and even more specifically from 1/30,000 to 1/40,000 (weight/weight), but it is not limited thereto.

In the present invention, the percentage of carboxypeptidase B relative to proinsulin is in the range from 1/600 to 1/20,000 (weight/weight), and specifically from 1/600 to 1/15,000 (weight/weight), but it is not limited thereto.

In particular, when both trypsin and carboxypeptidase B are used, the ratios of trypsin and carboxypeptidase B described above may be appropriately combined and used

The pH in the enzymatic conversion of the present invention may not be particularly limited as long as an effective conversion of proinsulin into insulin is possible, and specifically in the range from 6.5 to 9.0, and specifically from 7.0 to 8.5, but it is not limited thereto.

In the present invention, the temperature in the enzyme reaction may be in the range from 4.0° C. to 25.0° C., but it is not limited thereto.

In the present invention, the reaction time may be in the range from 4.0 hours to 55 hours, but it is not limited thereto.

In the present invention, the buffer in the enzyme reaction may be in the range from 1 mM to 100 mM Tris-HCl, but it is not limited thereto.

In the present invention, the buffer in the enzyme reaction may not comprise the metal ion.

Herein below, proinsulin and insulin will be described in greater detail.

As used herein, the term “proinsulin” refers to a precursor molecule of insulin. The insulin may include an insulin A-chain and an insulin B-chain, and a C-peptide there between. The proinsulin may be human proinsulin.

As used herein, the term “insulin” refers to a protein which is involved in the regulation of blood glucose levels in vivo.

Native insulin is a hormone secreted by the pancreas, which generally plays a role in regulating in vivo blood glucose levels by promoting the absorption of intracellular glucose while inhibiting fat cleavage.

Insulin, in the form of proinsulin without blood glucose level-regulating capability, is processed into insulin with blood glucose level-regulating capability. Insulin is composed of 2 polypeptide chains, i.e., the A-chain and the B-chain, which include 21 amino acids and 30 amino acids, respectively, and are interlinked by a disulfide bridge. Each of the A-chain and the B-chain may include the amino acid sequences represented by SEQ ID NO: 1 and SEQ ID NO: 2 shown below.

A-chain:
(SEQ ID NO: 1)
Gly-Ile-Val-Glu-Gln-Cys-Cys-Thr-Ser-Ile-Cys-Ser-
Leu-Tyr-Gln-Leu-Glu-Asn-Tyr-Cys-Asn
B-chain:
(SEQ ID NO: 2)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

In the present invention, proinsulin and insulin are conceived as including both native insulin and those in the form of insulin analogs.

In the present invention, proinsulin analogs or insulin analogs include those where amino acids in the B-chain or A-chain are modified, compared with those of native types. The insulin analogs may possess in vivo blood glucose level-controlling capability equivalent or corresponding to that of native insulin.

Specifically, the proinsulin analogs or insulin analogs may include those where at least one amino acid in native insulin is modified by any one selected from the group consisting of substitution, addition, deletion, modification, and a combination thereof, but they are not limited thereto.

The insulin analogs used in Examples of the present invention were prepared by genetic recombination technology, and these insulin analogs include the concepts of inverted insulin, insulin variants, insulin fragments, etc.

These insulin analogs, being peptides having in vivo blood glucose level-controlling capability equivalent or corresponding to that of native insulin, include all the concepts of insulin agonists, insulin derivatives, insulin fragments, insulin variants, etc.

The insulin derivatives have the in vivo blood glucose level-controlling capability, have a homology to each of the amino acid sequences of the A-chain and B-chain of native insulin, and include peptides in the forms where a part of the groups in amino acid residues is modified by a chemical substitution (e.g., alpha-methylation, alpha-hydroxylation), deletion (e.g., deamination), or modification (e.g., N-methylation). These insulin fragments refer to those in the forms where at least one amino acid is either inserted or deleted to insulin, and the inserted amino acid(s) may be those which are not present in nature (e.g., a D-type amino acid), and these insulin fragments possess the in vivo blood glucose level-controlling capability.

These insulin variants, being peptides where at least one amino acid sequence differs from that of insulin, possess the in vivo blood glucose level-controlling capability.

The methods used in preparing the insulin agonists, insulin derivatives, insulin fragments, and insulin variants of the present invention may be independently used or combined as well. For example, the peptides having the in vivo blood glucose level-controlling capability, where at least one amino acid sequence differs from that of insulin and deamination is introduced to the N-terminal amino acid residue, are also included in the scope of the present invention.

Specifically, the proinsulin analogs or insulin analogs may be those where at least one amino acid selected from the group consisting of amino acids of the B-chain at positions 1, 2, 3, 5, 8, 10, 12, 16, 23, 24, 25, 26, 27, 28, 29, and 30; and amino acids of the A-chain at positions 1, 2, 5, 8, 10, 12, 14, 16, 17, 18, 19, and 21; and more specifically, those, where at least one amino acid selected from the group consisting of amino acids of the B-chain at positions 8, 16, 23, 24, and 25; and amino acids of the A-chain at positions 1, 2, 14, and 19, may be substituted with another amino acid. Specifically, in the above amino acids, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, or 27 or more amino acids may be substituted with another amino acid(s), but are not limited thereto.

The amino acid residues at positions described above may also be substituted with alanine, glutamic acid, asparagine, isoleucine, valine, glutamine, glycine, lysine, histidine, cysteine, phenylalanine, tryptophan, proline, serine, threonine, and/or aspartic acid. For example, the amino acid at the 14^{th} position of the A-chain of native insulin, i.e., tyrosine, may be substituted with glutamic acid.

For the substitution or insertion of the amino acids, not only the 20 amino acids conventionally observed in human proteins but also atypical or unnatural amino acids may be used. The atypical amino acids may be commercially obtained from Sigma-Aldrich, ChemPep, Genzymepharmaceuticals, etc. The peptides containing these amino acids and typical peptide sequences may be synthesized by or purchased from the commercial companies, such as peptide synthesis companies American Peptide Company, Bachem (USA), and Anygen (Korea).

More specifically, the insulin analogs may be those which include the A-chain of SEQ ID NO: 3 represented by the following General Formula 1 and/or the B-chain of SEQ ID NO: 4 represented by the following General Formula 2, and additionally, the A-chain and the B-chain may be interlinked by a disulfide bond, but are not limited thereto.

[General Formula 1]
(SEQ ID NO: 3)
Xaa1-Xaa2-Val-Glu-Gln-Cys-Cys-Thr-Ser-Ile-Cys-Ser-
Leu-Xaa3-Gln-Leu-Glu-Asn-Xaa4-Cys-Asn

In General Formula 1,

Xaa1 is glycine or alanine,

Xaa2 is isoleucine or alanine,

Xaa3 is tyrosine, glutamic acid, asparagine, histidine, lysine, alanine, or aspartic acid, and

Xaa4 is tyrosine, glutamic acid, serine, threonine or alanine.

[General Formula 2]
(SEQ ID NO: 4)
Phe-Val-Asn-Gln-His-Leu-Cys-Xaa5-Ser-His-Leu-Val-
Glu-Ala-Leu-Xaa6-Leu-Val-Cys-Gly-Glu-Arg-Xaa7-Xaa8-
Xaa9-Tyr-Thr-Pro-Lys-Thr

In General Formula 2,

Xaa5 is glycine or alanine,

Xaa6 is Tyrosine, glutamic acid, serine, threonine or aspartic acid

Xaa7 is glycine or alanine,

Xaa8 is phenylalanine or alanine, and

Xaa9 is phenylalanine aspartic acid, glutamic acid alanine or deletion.

More specifically, the insulin analogs may be those which include:

(i) an A-chain, wherein Xaa1 isalanine, Xaa2 is isoleucine, Xaa3 is tyrosine, and Xaa4 is tyrosine in General Formula 1; and a B-chain, wherein Xaa5 is glycine, Xaa6 is tyrosine, Xaa7 is glycine, Xaa8 is phenylalanine, and Xaa9 is phenylalanine in General Formula 2;

(ii) an A-chain, wherein Xaa1 is glycine, Xaa2 is alanine, Xaa3 is tyrosine, and Xaa4 is tyrosine in General Formula 1; and a B-chain, wherein Xaa5 is glycine, Xaa6 is tyrosine, Xaa7 is glycine, Xaa8 is phenylalanine, and Xaa9 is phenylalanine in General Formula 2;

(iii) an A-chain, wherein Xaa1 is glycine, Xaa2 is isoleucine, Xaa3 is glutamic acid asparagine, histidine, lysine, alanine or aspartic acid, and Xaa4 is tyrosine in General Formula 1; and a B-chain, wherein Xaa5 is glycine, Xaa6 is tyrosine, Xaa7 is glycine, Xaa8 is phenylalanine, and Xaa9 is phenylalanine in General Formula 2;

(iv) an A-chain, wherein Xaa1 is glycine, Xaa2 is isoleucine, Xaa3 is tyrosine, and Xaa4 is alanine, glutamic acid, serine or threonine in General Formula 1; and a B-chain, wherein Xaa5 is glycine, Xaa6 is tyrosine, Xaa7 is glycine, Xaa8 is phenylalanine, and Xaa9 is phenylalanine in General Formula 2;

(v) an A-chain, wherein Xaa1 is glycine, Xaa2 is isoleucine, Xaa3 is tyrosine, and Xaa4 is tyrosine in General Formula 1; and a B-chain, wherein Xaa5 is alanine, Xaa6 is tyrosine, Xaa7 is glycine, Xaa8 is phenylalanine, and Xaa9 is phenylalanine in General Formula 2;

(vi) an A-chain, wherein Xaa1 is glycine, Xaa2 is isoleucine, Xaa3 is tyrosine, and Xaa4 is tyrosine in General Formula 1; and a B-chain, wherein Xaa5 is glycine, Xaa6 is glutamic acid, serine, threonine or aspartic acid, Xaa7 is glycine, and Xaa8 is phenylalanine Xaa9 is phenylalanine in General Formula 2;

(vii) an A-chain, wherein Xaa1 is glycine, Xaa2 is isoleucine, Xaa3 is tyrosine, and Xaa4 is tyrosine in General Formula 1; and a B-chain, wherein Xaa5 is glycine, Xaa6 is tyrosine, Xaa7 is alanine, Xaa8 is phenylalanine, and Xaa9 is phenylalanine in General Formula 2;

(viii) an A-chain, wherein Xaa1 is glycine, Xaa2 is isoleucine, Xaa3 is tyrosine, and Xaa4 is tyrosine in General Formula 1; and a B-chain, wherein Xaa5 is glycine, Xaa6 is tyrosine, Xaa7 is glycine, Xaa8 is alanine, and Xaa9 is phenylalanine in General Formula 2;

(ix) an A-chain, wherein Xaa1 is glycine, Xaa2 is isoleucine, Xaa3 is tyrosine, and Xaa4 is tyrosine in General Formula 1; and a B-chain, wherein Xaa5 is glycine, Xaa6 is tyrosine, Xaa7 is glycine, Xaa8 is phenylalanine, and Xaa9 is alanine, aspartic acid or glutamic acid in General Formula 2;

(x) an A-chain, wherein Xaa1 is glycine, Xaa2 is isoleucine, Xaa3 is glutamic acid, and Xaa4 is tyrosine in General Formula 1; and a B-chain, wherein Xaa5 is glycine, Xaa6 is tyrosine, Xaa7 is glycine, Xaa8 is phenylalanine, and Xaa9 is deletion in General Formula 2; and

(xi) an A-chain, wherein Xaa1 is glycine, Xaa2 is isoleucine, Xaa3 is alanine, and Xaa4 is tyrosine in General Formula 1; and a B-chain, wherein Xaa5 is glycine, Xaa6 is glutamic acid, Xaa7 is glycine, Xaa8 is phenylalanine, and Xaa9 is deletion in General Formula 2, but are not limited thereto.

For example, those peptides, which include the characteristic amino acid sequences described above and have a sequence homology to the that of the corresponding insulin analog of at least 70%, specifically at least 80%, more specifically at least 90%, and even more specifically at least 95%, while having the blood glucose level-controlling capability, also belong to the scope of the present invention.

As used herein, the term “homology” refers to a degree of similarity with a given amino acid sequence of a native wild-type protein or a polynucleotide sequence encoding the same, and includes those sequences which have the identity of the above-described percentages to the amino acid sequences or polynucleotide sequences of the present invention. The homology may be determined by comparing the two given sequences by the naked eye or may be determined using a bioinformatic algorithm, which enables the analysis of a homology by arranging the subject sequences for comparison. The homology between the two given amino acid sequences may be indicated as a percentage. The useful automated algorithm is available for use in GAP, BESTFIT, FASTA, and TFASTA computer software modules of Wisconsin Genetics Software Package (Genetics Computer Group, Madison, Wis., USA).

The arrangement algorithm automated in the above modules includes sequence arrangement algorithm by Needleman & Wunsch, Pearson & Lipman, and Smith & Waterman. Other useful algorithms on sequence arrangement and homology determination are automated in software including FASTP, BLAST, BLAST2, PSIBLAST, and CLUSTAL W.

The insulin analogs may have modifications, such as A¹G→A, A²I→A, A¹⁹Y→A, B⁸G→A, B²³G→A, B²⁴F→A, B²⁵F→A, A¹⁴Y→E, A¹⁴Y→N, A¹⁴Y→H, A¹⁴Y→K, A¹⁹Y→E, A¹⁹Y→S, A¹⁹Y→T, B¹⁶Y→E, B¹⁶Y→S, B¹⁶Y→T, A¹⁴Y→A, A¹⁴Y→D, B¹⁶Y→D, B²⁵F→D, B²⁵F→E, A¹⁴Y→D/B²⁵F→deletion, and/or A¹⁴Y→D/B¹⁶Y→E/B²⁵F→deletion but are not limited thereto (In particular, A or B described in the initial character refers to the A-chain or B-chain of insulin, and the number described therein indicates the amino acid number in the corresponding chain. The final characters stand for abbreviated amino acids named according to the IUPAC, for example, G→A indicates that glycine was substituted with alanine.).

Examples of the insulin analogs to be applied to the present invention may not be limited to those described above, but various insulin analogs disclosed in the art may be applied to the method of the present invention.

Additionally, it would be easy for a skilled person in the art to design and apply these insulin analogs as proinsulin analogs.

The proinsulin used in the method of the present invention may be that which was expressed in a microorganism and then obtained by partial purification, but it is not limited thereto. In particular, the proinsulin may be that partially purified using a cation exchange column.

Specifically, the proinsulin may undergo a purification step, which includes (a) expressing the proinsulin in a microorganism in the form of an inclusion body followed by isolating the inclusion body therefrom; (b) refolding the proinsulin from the inclusion body containing the isolated proinsulin; and (c) purifying the proinsulin obtained in step (b).

For example, the purification may be performed by the following process.

Specifically, the proinsulin may be expressed and formed by fermentation in a microorganism in the form of an inclusion body. The cell membrane of the microorganism is crushed using a high-pressure microfluidizer in order to isolate the inclusion body formed within the microorganism. The microorganism where the cell membrane is crushed is subjected to centrifugation and washing, and only the inclusion body containing the proinsulin is isolated and obtained.

After reacting the insulin precursor protein with a reducing agent in a glycine buffer to reduce the disulfide bond of the insulin precursor protein contained in the thus-obtained inclusion body pellet, the resultant was added with a chaotropic agent to linearize the structure of the insulin precursor protein. Then, the remainder is removed by centrifugation, and the concentrations of the chaotropic agent and the reducing agent are lowered by diluting with distilled water, thereby enabling the formation of a protein having the accurate structure of the insulin precursor.

Subsequently, for the isolation of the accurately-formed proinsulin, cation exchange chromatography or anion exchange chromatography may be applied.

The method of the present invention may further include purifying the sample containing insulin, which was converted from proinsulin by enzymatic conversion.

Specifically, the method may be applied to insulin by subjecting the sample containing insulin converted from proinsulin to chromatography.

The chromatography may not be particularly limited as long as it enables an effective purification, and may be cation exchange chromatography or reversed phase chromatography.

As used herein, the term “cation exchange chromatography” refers to chromatography which utilizes a column filled with a cation exchange resin. The cation exchange resin is a synthetic resin which is added into a different aqueous solution and exchanges its own cations with the cations present in the aqueous solution. For the cation exchange resin, various resins conventionally used in the art may be used, and specifically, a column having the functional group of COO⁻ or SO₃²⁻, for example, those columns which have methanesulfonate (S), sulfopropyl (SP), carboxymethyl (CM), polyaspartic acid, sulfoethyl (SE), sulfopropyl (SP), phosphate (P), sulfonate (S), etc., may be used, although are not limited thereto.

The cation exchange chromatography may be performed by attaching insulin to a column by subjecting the sample to the equilibrated cation exchange column, and then eluting it therefrom using an elution buffer solution.

The equilibration of the cation exchange column may be performed using various buffer solutions, e.g., citrate, acetate, phosphate, MOPS or MES buffer solution, etc.

The elution buffer solution may be performed using various salt solutions, e.g., NaCl or KCl salt buffer solution. The elution may be performed using a linear concentration gradient, a stepwise concentration gradient, etc., but is not limited thereto.

Additionally, the purification of insulin may further include performing reversed phase chromatography after performing cation exchange chromatography.

As used herein, the term “reversed phase chromatography” refers to chromatography which enables separation of a mixture using a combination of the stationary phase with high polarity and the mobile phase with low polarity.

For the reversed phase chromatography resin, various resins which are conventionally used in the art may be used, and specifically the columns which have a functional group in the form of a carbon body in a silica or polymer matrix or the columns where the polymer matrix itself can act as a functional group, e.g., columns having C2, C4, C8, C18 or polystyrene/divinyl benzene, etc., may be used, although are not limited thereto.

The reversed phase chromatography may be performed by attaching insulin to a column by subjecting the sample to the equilibrated column, and then eluting the insulin therefrom using an elution buffer solution.

The equilibration of the reversed phase chromatography may be performed using various buffer solutions, e.g., phosphate, water containing TFA/TAE, etc.

The elution buffer solution may be performed using various organic solvents, e.g., ethanol, isopropanol, acetonitrile, etc. The above elution may be performed using a linear concentration gradient, a stepwise concentration gradient, etc., but is not limited thereto.

Additionally, the purification of proinsulin and insulin may further include performing anion exchange chromatography after performing cation exchange chromatography.

As used herein, the term “anion exchange chromatography” refers to chromatography which utilizes a column filled with an anion exchange resin. The anion exchange resin is a synthetic resin which is added into a different aqueous solution and exchanges its own anions with the anions present in the aqueous solution. For the anion exchange resin, various resins conventionally used in the art may be used, and specifically, a column having the functional group of N⁺, for example, those columns which have quaternary ammonium (Q), quaternary aminoethyl (QAE), diethylaminoethyl (DEAE), polyethyleneimine (PEI), dimethylaminoethyl (DMAE), trimethylaminoethyl (TMAE), etc., may be used, but are not limited thereto.

The anion exchange chromatography may be performed by attaching proinsulin and insulin to a column by subjecting the sample to the equilibrated anion exchange column, and then eluting them therefrom using an elution buffer solution.

The equilibration of the anion exchange column may be performed using various buffer solutions, e.g., Tris, bis-Tris, histidine, HEPES buffer solution, etc.

The elution buffer solution may be performed using various salt solutions, e.g., NaCl or KCl salt buffer solution. The elution may be performed using a linear concentration gradient, a stepwise concentration gradient, etc., but is not limited thereto.

Additionally, the proinsulin used for preparing insulin by enzymatic cleavage of the present invention may be partially purified by a cation exchange column or a reversed column.

Meanwhile, according to the method of the present invention, insulin may be prepared so that the content of Des-Thr(B30)-insulin impurities can be contained at less than 5%, specifically, less than 3%, more specifically less than 2%, and even more specifically less than 1%, although it is not particularly limited thereto.

In another aspect to achieve the above objects, the present invention provides a method of purifying insulin comprising preparing a sample containing insulin by converting high-concentration proinsulin into insulin by enzymatic cleavage; and subjecting the sample to a purification process.

The purification process may be conducted by a chromatography process.

The steps of preparing a sample containing insulin, steps of purifying the sample, and chromatography process are the same as described above.

In a further aspect to achieve the above objects, the present invention provides insulin prepared by the above method.

The above preparation method and insulin are the same as described above.

Hereinafter, the present invention will be described in more detail with reference to the following Examples. However, these Examples are for illustrative purposes only, and the invention is not intended to be limited by these Examples.

### Example 1: Expression of Proinsulin Analogs

The expression of recombinant proinsulin analogs was performed under the regulation of T7 promoter. The sequences corresponding to insulin of each analog are shown in Table 1 below.

TABLE 1

| | | | SEQ ID |
|---|---|---|---|
| Analog | Sequence | | NO |
| Analog 1 | DNA | TTC GTT AAC CAA CAC TTG TGT GGC TCA CAC CTG GTG GAA GCT | 5 |
| | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GCG ATT GTG GAA CAA | |
| | | TGC TGT ACC AGC ATC TGC TCC CTC TAC CAG GTG GAG AAC TAC | |
| | | TGC AAC | |
| | Protein | Phe Val Asn Gln His Leu Cys Gly Ser His Leu Val Glu Ala | 6 |
| | | Leu Tyr Leu Val Cys Gly Glu Arg Gly Phe Phe Tyr Thr Pro | |
| | | Lys Tnr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Ala Ile Val Glu Gln | |
| | | Cys Cys Thr Ser Ile Cys Ser Leu Tyr Gln Leu Glu Asn Tyr | |
| | | Cys Asn | |
| Analog 2 | DNA | TTC GTT AAC CAA CAC TTG TGT GGC TCA CAC CTG GTG GAA GCT | 7 |
| | | CTC TAC CTA GTG TGC GGG GAA CGA GGC TTC TTC TAC ACA CCC | |
| | | AAG ACC CCC CCC GAG GCA GAG GAC CTG CAG GTG GGG CAG GTG | |
| | | GAG CTG GGC GGG GGC CTT GGT GCA GGC AGC CTG CAG CCC TTG | |
| | | GCC CTG GAG GGG TCC CTG CAG AAG CGT GGC GCG GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 8 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ala Val Glu Gln | |
| | | Cys Cys Thr Ser Ile Cys Ser Leu Tyr Gln Leu Glu Asn Tyr | |
| | | Cys Asn | |
| Analog 3 | DNA | TTC GTT AAC CAA CAC TTG TGT GGC TCA CAC CTG GTG GAA GCT | 9 |
| | | CTC TAC CTA GTG TGC GGG GAA CGA GGC TTC TTC TAC ACA CCC | |
| | | AAG ACC CCC CCC GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC GCG | |
| | | TGC AAC | |
| | Protein | Phe Val Asn Gln His Leu Cys Gly Ser His Leu Val Glu Ala | 10 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Ala | |
| | | Cys Asn | |
| Analog 4 | DNA | TTC GTT AAC CAA CAC TTG TGT GCG TCA CAC CTG GTG GAA GCT | 11 |
| | | CTC TAC CTA GTG TGC GGG GAA CGA GGC TTC TTC TAC ACA CCC | |
| | | AAG ACC CCC CCC GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Ala Ser His Leu Val Glu Ala | 12 |
| | | Leu Tyr Lea Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog 5 | DNA | TTC GTT AAC CAA CAC TTG TGT GGC TCA CAC CTG GTG GAA GCT | 13 |
| | | CTC TAC CTA GTG TGC GGG GAA CGA GCG TTC TTC TAC ACA CCC | |
| | | AAG ACC CGC CCC GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 14 |
| | | Leu Tyr Leu Val Cys Gly Glu Arg Ala Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog 6 | DNA | TTC GTT AAC CAA CAC TTG TGT GGC TCA CAC CTG GTG GAA GCT | 15 |
| | | CTC TAC CTA GTG TGC GGG GAA CGA GGC GCG TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 16 |
| | | Leu Tyr Leu Val Cys Gly Glu Arg Gly Ala Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Ary Gly Ile Val Glu Gln | |
| | | Cys Cys Thr Ser Ile Cys Ser Leu Tyr Gln Leu Glu Asn Tyr | |
| | | Cys Asn | |
| Analog 7 | DNA | TTC GTT AAC CAA CAC TTG TGT GGC TCA CAC CTG GTG GAA GCT | 17 |
| | | CTC TAC CTA GTG TGC GGG GAA CGA GGC TTC GCG TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 18 |
| | | Leu Tyr Leu Val Cys Gly Glu Arg Gly Phe Ala Tyr Thr Pro | |
| | | Lys Thr Arg Arg Glu Ala Glu Asp Leu Gln Val Gly Gln Val | |
| | | Glu beu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog 8 | DNA | TTC GTT AAC CAA CAC TTG TGT GGC TCA CAC CTG GTG GAA GCT | 19 |
| | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC GAA CAG CTG GAG AAC TAC | |
| | | TGC AAC TGA | |
| | Protein | Phe Val Asn Gln His Leu Cys Gly Ser His Leu Val Glu Ala | 20 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Glu Gln Leu Glu Asn Tyr | |
| | | Cys Asn | |
| Analog 9 | DNA | TTC GTT AAC CAA CAC TTG TGT GGC TCA CAC CTG GTG GAA GCT | 21 |
| | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC AAC CAG CTG GAG AAC TAC | |
| | | TGC AAC TGA | |
| | Protein | Phe Val Asn Gln His Leu Cys Gly Ser His Leu Val Glu Ala | 22 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Asn Gln Leu Glu Asn Tyr | |
| | | Cys Asn | |
| Analog | DNA | TTC GTT AAC CAA CAC TTG TGT GGC TCA CAC CTG GTG GAA GCT | 23 |
| 10 | | CTC TAC CTA GTG TGC GGG GAA CGA GGC TTC TTG TAC ACA CCC | |
| | | AAG ACC CGC CGG GAG GCA GAG GAC CTG CAG GTG GGG CAG GTG | |
| | | GAG CTG GGC GGG GGG CCT GGT GCA GGC AGC CTG CAG CCC TTC | |
| | | GCC CTG GAG GGG TCC CTG CAG AAG CGT GGC ATT GTG GAA CAA | |
| | | TGC TGT ACC AGC ATC TGC TCC CTC CAT CAG CTG GAG AAC TAC | |
| | | TGC AAC | |
| | Protein | Phe Val Asn Gln His Leu Cys Gly Ser His Leu Val Glu Ala | 24 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gay Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu His Gln Leu Glu Asn Tyr | |
| | | Cys Asn | |
| Analog | DNA | TTG GTT AAC CAA CAC TTG TGT GGC TCA CAC CTG GTG GAA GCT | 25 |
| 11 | | CTC TAC CTA GTG TGC GGG GAA CGA GGC TTC TTC TAG ACA CCC | |
| | | AAG ACC CGC CGG GAG GCA GAG GAC CTG CAG GTG GGG GAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC AAG CAG CTG GAG AAC TAG | |
| | | TGC AAC | |
| | Protein | Phe Val Asn Gln His Leu Cys Gly Ser His Leu Val Glu Ala | 26 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Lys Gln Leu Glu Asn Tyr | |
| | | Cys Asn | |
| Analog | DNA | TTC GTT AAC CAA CAC TTG TGT GGC TCA CAC CTG GTG GAA GCT | 27 |
| 12 | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG GAG AAG CGT GGC ATT GTG GAA CAA | |
| | | TGC TGT ACC AGC ATC TGC TCC CTC TAC CAG CTG GAG AAC GAG | |
| | | TGC AAC | |
| | Protein | Phe Val Asn Gln His Leu Cys Gly Ser His Leu Val Glu Ala | 26 |
| | | Leu Tyr Lea Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Glu | |
| | | Cys Asn | |
| Analog | DNA | TTC GTT AAG CAA CAC TTG TGT GGC TCA CAC CTG GTG GAA GCT | 29 |
| 13 | | CTC TAC CTA GTG TGC GGG GAA CGA GGC TTC TTC TAC ACA CCC | |
| | | AAG ACC CGC CCC GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG AGT GGC ATT GTG GAA CAA | |
| | | TGC TGT ACC AGC ATC TGC TCC CTC TAC CAG CTG GAG AAC TCC | |
| | | TGC AAC | |
| | Protein | Phe Val Asn Gln His Leu Cys Gly Ser His Leu Val Glu Ala | 30 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Ser | |
| | | Cys Asn | |
| Analog | DNA | TTC GTT AAC CAA CAC TTG TGT GGC TCA CAC CTG GTG GAA GCT | 31 |
| 14 | | CTC TAC CTA GTG TGC GGG GAA CGA GGC TTC TTC TAC ACA CCC | |
| | | AAG ACC CCC CCC GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC ACC | |
| | | TGC AAC | |
| | Protein | Phe Val Aan Gln His Leu Cys Gly Ser His Leu Val Glu Ala | 32 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Thr | |
| | | Cys Asn | |
| Analog | DNA | TTC GTT AAC CAA CAC TTG TGT GGC TCA CAC CTG GTG GAA GCT | 33 |
| 15 | | CTC GAG CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 34 |
| | | Leu Glu Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog | DNA | TTC GTT AAC CAA CAC TTG TGT GGC TCA CAC CTG GTG GAA GCT | 35 |
| 16 | | CTC TCC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 36 |
| | | Leu Ser Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog | DNA | TTC GTT AAC CAA CAC TTG TGT GGC TCA CAC CTG GTG GAA GCT | 37 |
| 17 | | CTC ACC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asa Gln His Leu Cys Gly Ser His Leu Val Gln Ala | 38 |
| | | Leu Thr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Gln Gly Ser Leu Gln Lys Arg Gly Ile Val Gln Gln | |
| | | Cys Cys Thr Ser Ile Cys Ser Leu Tyr Gln Leu Glu Asn Tyr | |
| | | Cys Asn | |
| Analog | DNA | TTC GTT AAC CAA CAC TTG TGT GGC TCA CAC CTG GTG GAA GCT | 39 |
| 18 | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TTC CTG CAG AAG CGT GGC ATT GTG GAA CAA | |
| | | TGC TGT ACC AGC ATC TGC TCC CTC GCC CAG CTG GAG AAC TAC | |
| | | TGC AAC | |
| | Protein | Phe Val Asn Gln His Leu Cys Gly Ser His Leu Val Gln Ala | 40 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Ala Gln Leu Glu Asn Tyr | |
| | | Cys Asn | |
| Analog | DNA | TTC GTT AAC CAA CAC TTG TGT GGC TCA CAC CTG GTG GAA GCT | 41 |
| 19 | | CTC TAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC GAC CAG CTG GAG AAC TAC | |
| | | TGC AAC | |
| | Protein | Phe Val Asn Gln His Leu Cys Gly Ser His Leu Val Glu Ala | 42 |
| | | Leu Tyr Leu Val Cys Gly Glu Arg Gly Phe Phe Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Asp Gln Leu Glu Asn Tyr | |
| | | Cys Asn | |
| Analog | DNA | TTC GTT AAC CAA CAC TTG TGT GGC TCA CAC CTG GTG GAA GCT | 43 |
| 20 | | CTC GAC CTA GTG TGC GGG GAA CGA GGC TTC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Gln Ala | 44 |
| | | Leu Asp Leu Val Cys Gly Glu Arg Gly Phe Phe Tyr Thr Pro | |
| | | Lys Thr Arg Arg Gln Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Gln Asn Tyr | |
| | | Cys Asn | |
| Analog | DNA | TTC GTT AAC CAA CAC TTG TGT GGC TCA CAC CTG GTG GAA GCT | 45 |
| 21 | | CTC TAC CTA GTG TGC GGG GAA CGA GGC TTC GAC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAG CAG CTG GAG AAC TAC | |
| | | TGC AAC | |
| | Protein | Phe Val Asn Gln His Leu Cys Gly Ser His Leu Val Glu Ala | 46 |
| | | Leu Tyr Leu Val Cys Gly Glu Arg Gly Phe Asp Tyr Thr Pro | |
| | | Lys Thr Arg Arg Gln Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Gln Asn Tyr | |
| | | Cys Asn | |
| Analog | DNA | TTC GTT AAC CAA CAC TTG TGT GGC TCA CAC CTG GTG GAA GCT | 47 |
| 22 | | CTC TAC CTA GTG TGC GGG GAA CGA GGC TTC GAG TAC ACA CCC | |
| | | AAG ACC CCC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC TAC CAG CTG GAG AAC TAC | |
| | | TGC AAC | |
| | Protein | Phe Val Asn Gln His Leu Cys Gly Ser His Leu Val Glu Ala | 48 |
| | | Leu Tyr Leu Val Cys Gly Glu Arg Gly Phe Glu Tyr Thr Pro | |
| | | Lys Thr Arg Arg Glu Ala Glu Asp Leu Gln Val Gly Gln Val | |
| | | Glu Leu Gly Gly Gly Pro Gly Ala Gly Ser Leu Gln Pro Leu | |
| | | Ala Leu Glu Gly Ser Leu Gln Lys Arg Gly Ile Val Glu Gln | |
| | | Cys Cys Thr Ser Ile Cys Ser Leu Tyr Gln Leu Glu Asn Tyr | |
| | | Cys Asn | |
| Analog | DNA | TTC GTT AAC CAA CAC TTG TGT GGC TCA CAC CTG GTG GAA GCT | 49 |
| 23 | | CTC TAC CTA GTG TGC GGG GAA CGA GGC TTC TAC ACA CCC | |
| | | AAG ACC CGC CGG GAG GCA GAG GAC CTG CAG GTG GGG CAG GTG | |
| | | GAG CTG GGC GGG GGC CCT GGT GCA GGC AGC CTG CAG CCC TTG | |
| | | GCC CTG GAG GGG TCC CTG CAG AAG CGT GGC ATT GTG GAA CAA | |
| | | TGC TGT ACC AGC ATC TGC TCC CTC GAA CAG CTG GAG AAC TAG | |
| | | TGC AAC | |
| | Protein | Phe Val Asn Gln His Leu Cys Gly Ser His Leu Val Glu Ala | 50 |
| | | Leu Tyr Leu Val Cys Gly Glu Arg Gly Phe Tyr Thr Pro Lys | |
| | | Thr Arg Arg Glu Ala Glu Asp Leu Gln Val Gly Gln Val Glu | |
| | | Leu Gly Gly Gly Pro Gly Ala Gly Ser Leu Gln Pro Leu Ala | |
| | | Leu Glu Gly Ser Leu Gln Lys Arg Gly Ile Val Glu Gln Cys | |
| | | Cys Thr Ser Ile Cys Ser Leu Glu Gln Leu Glu Asn Tyr Cys | |
| | | Asn | |
| Analog | DNA | TTC GTT AAC CAA CAC TTG TGT GGC TCA CAC CTG GTG GAA GCT | 51 |
| 24 | | CTC GAG CTA GTG TGC GGG GAA CGA GGC TTC TAC ACA CCC AAG | |
| | | ACC CCC CGG GAG GCA GAG GAC CTG CAG GTG GGG CAG GTG GAG | |
| | | CTG GGC GGG GGC CCT GGT GCA GGC AGC CTG CAG CCC TTG GCC | |
| | | CTG GAG GGG TCC CTG CAG AAG CGT GGC ATT GTG GAA CAA TGC | |
| | | TGT ACC AGC ATC TGC TCC CTC GCC CAG CTG GAG AAC TAC TGC | |
| | | AAC | |
| | Protein | Phe Val Asn Gln His Leu Cys Gly Ser His Leu Val Glu Ala | 52 |
| | | Leu Glu Leu Val Cys Gly Glu Arg Gly Phe Tyr Thr Pro Lys | |
| | | Thr Arg Arg Glu Ala Glu Asp Leu Gln Val Gly Gln Val Glu | |
| | | Leu Gly Gly Gly Pro Gly Ala Gly Ser Leu Gln Pro Leu Ala | |
| | | Leu Glu Gly Ser Leu Gln Lys Arg Gly Ile Val Glu Gln Cys | |
| | | Cys Thr Ser Ile Cys Ser Leu Ala Gln Leu Glu Asn Tyr Cys | |
| | | Asn | |

E. coli BL21-DE3 ((E. coli B F-dcm ompT hsdS(rB-mB-) gal ADE3); Novagen) was transformed with an expression vector for each recombinant insulin analog. The transformation was performed according to the method recommended by Novagen. Single E. coli colonies transformed with each of the recombinant expression vectors were inoculated into 2× Luria broth (LB) medium containing ampicillin (50 μg/mL) and cultured at 37° C. for 15 hours. The recombinant E. coli culture and the 2× LB medium were mixed at a 1:1 (v/v) ratio, and the mixture was respectively aliquoted in an amount of 1 mL into a cryo-tube, and stored at −140° C. The resultants were used as cell stocks for producing recombinant proinsulin protein.

For the expression of recombinant proinsulin analogs, 1 vial of each of the cell stocks was inoculated into 500 mL of 2× Luria broth and cultured with shaking water bath at 37° C. for 10 hours to 18 hours. The resulting cultures, collected in the amount of 200 mL, were respectively inoculated into two flasks containing 500 mL of fresh 2× Luria broth, and cultured with shaking water bath at 37° C. for 1 hour to 5 hours. The resultants were used as stock cultures. The stock cultures were inoculated into 17 L of a fermentation medium using a 50 L fermenter (MSJ-U2, B. E. MARUBISHI, JAPAN) and subjected to initial batch fermentation. The culture conditions were: 37° C., air supply of 20 L/min (1 vvm), stirring speed of 500 rpm, and pH 6.70 adjusted using 30% ammonia water. The fermentation proceeded by a fed-batch culture after adding a feeding solution when the nutrients in the medium were limited. The growth of bacteria was monitored based on OD values, and IPTG, at a final concentration of 500 μM, was introduced when an OD value reached 100 or higher. The cultivation was continued further for about 20 hours to 25 hours after the introduction. Upon completion of the cultivation, overexpressed proinsulin analogs were confirmed by SDS PAGE. The recombinant bacteria having an overexpression of proinsulin analogs were collected by centrifugation and stored at −80° C. prior to use.

### Example 2: Recovery and Refolding of Recombinant Proinsulin Analogs

In order to change the recombinant proinsulin analogs expressed in Example 1 to a soluble form, the cells were crushed and the analogs were refolded. The cell pellets, in the amount of 170 g (wet weight), were respectively resuspended in 1 L of a solubilizing buffer solution (50 mM Tris-HCl (pH 9.0), 1 mM EDTA (pH 8.0), 0.2 M NaCl, and 0.5% Triton X-100). The cells were crushed using M-110EH (Model M1475C, AC Technology Corp.), a microfluidizer processor, under a pressure of 15,000 psi. The crushed cell lysates were centrifuged at 4° C. at 12,000 g for 30 minutes and the supernatant discarded, and the pellets were respectively resuspended in 1 L of a washing buffer solution (0.5% Triton X-100, 50 mM Tris-HCl (pH 8.0), 0.2 M NaCl, and 1 mM EDTA). The resultants were centrifuged at 4° C. at 12,000 g for 30 minutes and the pellets were respectively resuspended in distilled water, and centrifuged in the same manner. The pellets were collected and resuspended in 600 mL of a buffer solution (1 M glycine and 3.78 g of Cysteine-HCl (pH 10.6)) and stirred at room temperature for 1.5 hours. The resuspended recombinant proinsulin analogs were collected by being charged with urea, and then stirred at room temperature. For the refolding of the solubilized recombinant proinsulin analogs, they were centrifuged at 4° C. for 40 minutes. The resulting supernatants were respectively recovered and stirred at from 4° C. to 8° C. for at least 17 hours while being adding into 3 L to 12 L of distilled water using a peristaltic pump.

### Example 3: Purification by Cation Exchange Chromatography

A sample where the refolding was completed was attached to an SP-FF (GE healthcare, USA) column, which was equilibrated using a 20 mM sodium citrate (pH 3.0) buffer solution containing ethanol, and then the proinsulin analog protein was eluted by a linear concentration gradient from 0% to 100% using a 20 mM sodium citrate (pH 3.0) buffer solution containing 0.5 mM potassium chloride and ethanol.

### Example 4: Conversion of Proinsulin into Insulin by Enzyme Treatment

The No. 8 analog among the analogs listed in Example 1 was used as a representative sample for the experiment of converting proinsulin into insulin by enzyme treatment. The proinsulin analog sample eluted by the SP-FF column was adjusted to have a final pH from 7.0 to 8.5, and concentrated to have a protein concentration from 5 mg/mL to 300 mg/mL. The enzyme reaction was performed according to the manufacturer's protocol. The protein sample was added in the 50 mM Tris-HCl with trypsin (Roche, Germany), which corresponds to a weight/weight ratio of about 1/3,900 to 1/62,400, and carboxypeptidase B (Roche, Germany), which corresponds to a weight/weight ratio of about 1/644 to 1/19,300 relative to the protein amount of the sample, and stirred at about 4° C. to 25° C. for 0 hours to 55 hours. To terminate the enzyme reaction, the pH was lowered to 3.5 or below.

### Example 5: Confirmation of Impurities Reducing Effect in Enzymatic Conversion Method

In Example 4, the optimized high-concentration condition was established in order to minimize the Des-Thr(B30)-insulin analog impurities. Proinsulin at concentrations of 5 mg/mL, 50 mg/mL, 100 mg/mL, 200 mg/mL, and 300 mg/mL were charged with trypsin, which corresponds to a weight/weight ratio of about 1/3,900 to 1/62,400, and carboxypeptidase B, which corresponds to a weight/weight ratio of about 1/644 to 1/19,300 relative to the protein amount of the sample, and the stirring and the termination of the reaction were performed in the same manner as in Example 4. The Des-Thr(B30)-insulin impurity content at each concentration was confirmed by RP-HPLC (C4) analysis.

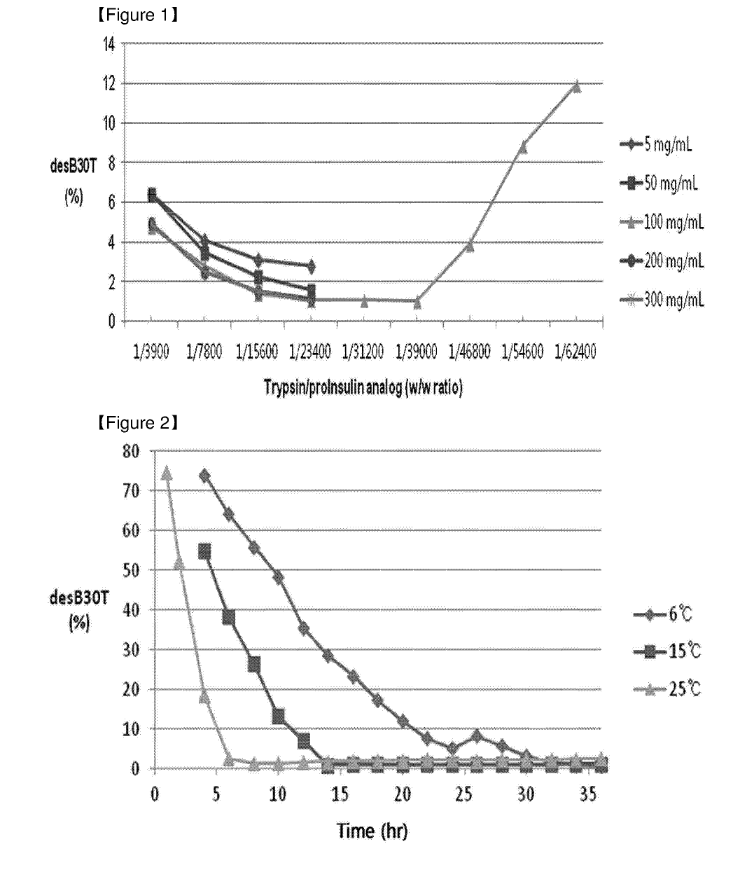

As a result, the Des-Thr(B30)-insulin analog impurities, when treated with trypsin (corresponding to a weight/weight ratio of about 1/3,900 to 1/62,400) and carboxypeptidase B (corresponding to a weight/weight ratio of about 1/644 relative to the protein amount) of the sample, were shown to occur at about 1.6% to 6.4% at 5 mg/mL to 50 mg/mL of proinsulin, whereas the occurrence rate was significantly lowered to about 1% at 100 mg/mL to 300 mg/mL of proinsulin, implying a significant inhibition (Table 2 and FIG. 1).

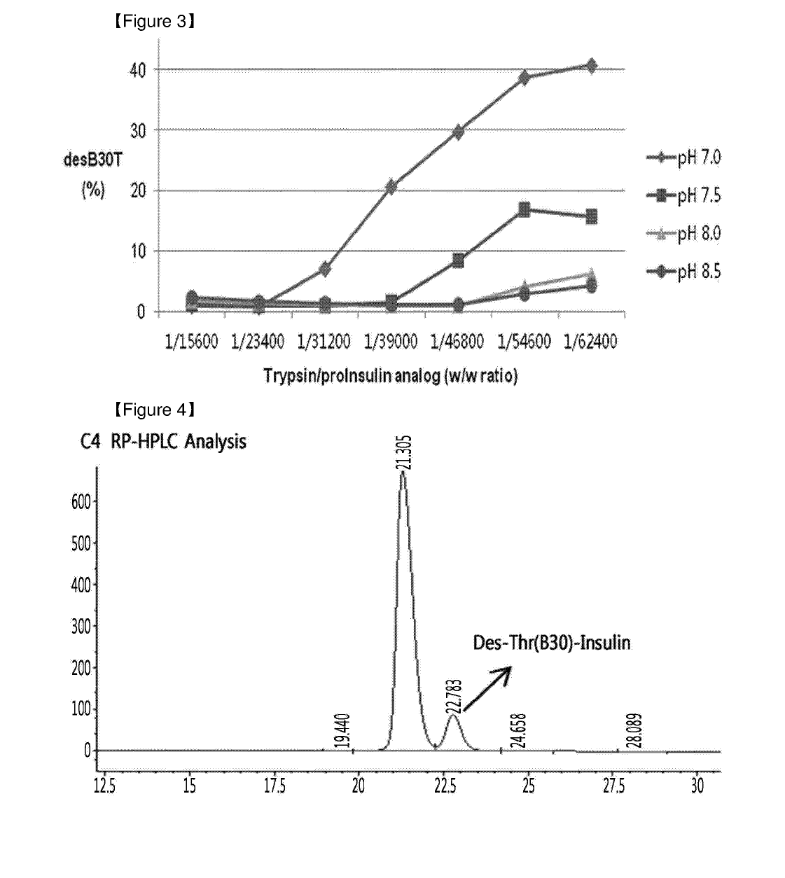

Experiments were performed from the optimized time of 0 hours to 36 hours or more (max. 55 hours) according to the reaction temperature, and the conditions for each temperature are shown in FIG. 2. As a result, it was confirmed that the reaction speed varied from low temperature to room temperature, although the reducing effect remained the same. The optimal conditions for trypsin according to pH conditions were confirmed and the results are shown in FIG. 3.

TABLE 2

| Percentage of | Percentage of | Content of Des-Thr(B30)-insulin impurities according to | | | | |
|---|---|---|---|---|---|---|
| trypsin | carboxypeptidase B | proinsulin analog concentration (mg/mL) (area, %) | | | | |
| treatment^{a} | treatment^{b} | 5 | 50 | 100 | 200 | 300 |
| 1/3,900 | 1/644 | 6.44 | 6.41 | 4.79 | 4.94 | 4.94 |
| 1/7,800 | 1/644 | 4.11 | 3.46 | 2.78 | 2.52 | 2.52 |
| 1/15,600 | 1/644 | 3.10 | 2.23 | 1.41 | 1.54 | 1.54 |
| 1/23,400 | 1/644 | 2.81 | 1.56 | 1.10 to 1.25 | 1.05 | 1.05 |
| 1/31,200 | 1/644 | — | — | 1.05 to 1.11 | — | — |
| 1/39,000 | 1/644 | — | — | 0.91 to 1.14 | — | — |
| 1/46,800 | 1/644 | — | — | 3.90 | — | — |
| 1/54,600 | 1/644 | — | — | 8.88 | — | — |
| 1/62,400 | 1/644 | — | — | 11.92 | — | — |

^{a}Trypsin/Protein (weight/weight ratio),
^{b}CpB/Protein (weight/weight ratio)

Additionally, it was confirmed that the generation of Des-Thr(B30)-insulin analog impurities was also controlled by the weight ratio of carboxypeptidase B.

It was confirmed that, when treated with trypsin (corresponding to a weight ratio of 1/31,200) and carboxypeptidase B (corresponding to a weight ratio from 1/644 to 1/19,300 relative to the protein amount of the sample), the impurity occurrence rate was about 1% at a high concentration of 100 mg/mL, thus suggesting that the occurrence of the Des-Thr(B30)-insulin analog impurities can be inhibited (Table 3).

TABLE 3

| Percentage of | Percentage of | Des-Thr(B30)-insulin analog |
|---|---|---|
| trypsin | carboxypeptidase B | impurities content (area, %) |
| treatment^{a} | treatment^{b} | 100 mg/mL |
| 1/31,200 | 1/644 | 0.99 |
| | 1/965 | 0.92 |
| | 1/1,930 | 1.12 |
| | 1/2,895 | 0.96 |
| | 1/3,860 | 1.03 |
| | 1/4,825 | 1.10 |
| | 1/5,790 | 1.20 |
| | 1/6,755 | 1.12 |
| | 1/7,720 | 1.30 |
| | 1/8,685 | 1.57 |
| | 1/9,650 | 1.23 |
| | 1/14,475 | 1.78 |
| | 1/19,300 | 3.72 |

^{a}Trypsin/Protein (weight/weight ratio),
^{b}CpB/Protein (weight/weight ratio)

### Example 6: Cation Exchange Chromatography Purification

A sample, upon termination of a reaction, was reattached to an SP-HP (GE healthcare, USA) column, which was equilibrated with a 20 mM sodium citrate (pH 3.0) buffer solution, and the insulin analog protein was eluted by a linear concentration gradient using a 20 mM sodium citrate (pH 3.0) buffer solution containing 0.5 M KCl and ethanol.

### Example 7: Reversed Phase Chromatography Purification

In order to purely separate the insulin analog from the sample obtained in Example 6 after attachment to the reversed phase chromatography Source30 RPC (GE healthcare, USA), which was equilibrated with a buffer containing sodium phosphate and isopropanol, the insulin analog was eluted by a linear concentration gradient using a buffer solution containing sodium phosphate and isopropanol.

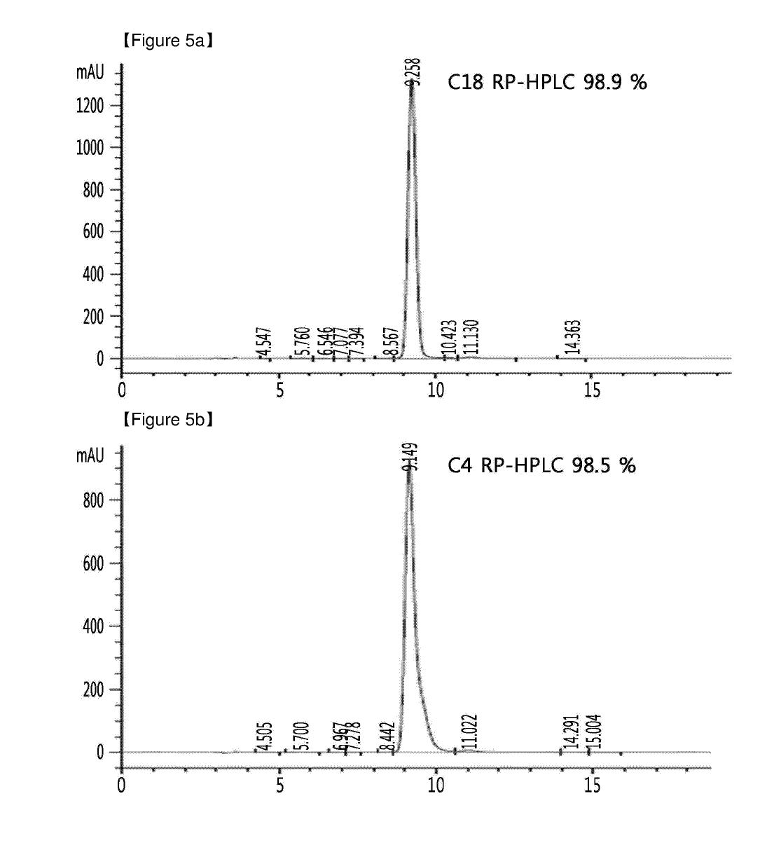

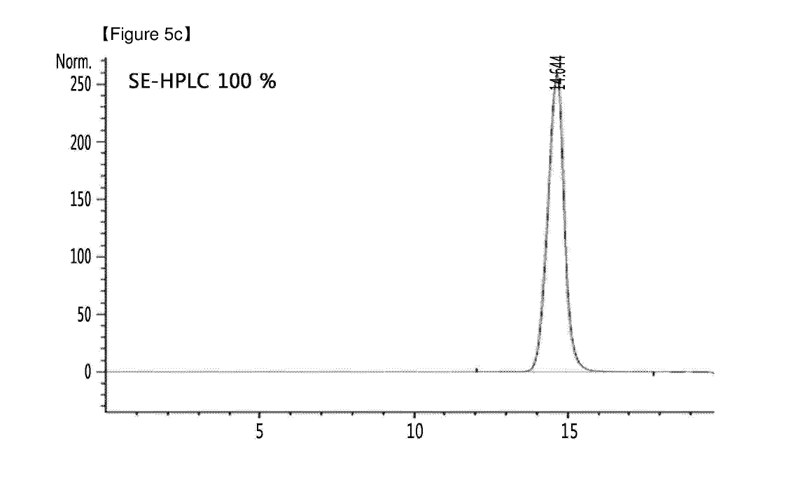

The insulin analog containing an excess amount (about 10%) of the Des-Thr(B30)-insulin analog was analyzed by HPLC (FIG. 4). The purity of the final insulin analog, which was purified by applying the enzymatic conversion to minimize the Des-Thr(B30)-insulin impurities, and the impurities were confirmed by HPLC analysis (FIG. 5). As a result, it was shown that the Des-Thr(B30)-insulin analog as a main impurity and the insulin analog in the form of deamination were about less than 1%, respectively, and the total purity was 98.5% or higher.

Those of ordinary skill in the art will recognize that the present invention may be embodied in other specific forms without departing from its spirit or essential characteristics. The described embodiments are to be considered in all respects only as illustrative and not restrictive. The scope of the present invention is, therefore, indicated by the appended claims rather than by the foregoing description. All changes which come within the meaning and range of equivalency of the claims are to be embraced within the scope of the present invention.

1.-26. (canceled)

27. A method of preparing insulin from proinsulin comprising converting proinsulin at a concentration of 50 mg/mL or higher into insulin by enzymatic cleavage.

28. The method of claim 27, wherein the enzyme is trypsin, carboxypeptidase B, or a combination thereof.

29. The method of claim 27, wherein the concentration of the proinsulin is in the range from 50 mg/mL to 300 mg/mL.

30. The method of claim 29, wherein the concentration of the proinsulin is in the range from 100 mg/mL to 300 mg/mL; or
wherein the concentration of the proinsulin is in the range from 200 mg/mL to 300 mg/mL.

31. The method of claim 28, wherein the percentage of trypsin relative to proinsulin is in the range from 1/7,500 to 1/40,000 (weight/weight);
wherein the percentage of trypsin relative to proinsulin is in the range from 1/15,000 to 1/40,000 (weight/weight);
wherein the percentage of trypsin relative to proinsulin is in the range from 1/20,000 to 1/40,000 (weight/weight); or
wherein the percentage of trypsin relative to proinsulin is in the range from 1/30,000 to 1/40,000 (weight/weight).

32. The method of claim 28, wherein the percentage of carboxypeptidase B relative to proinsulin is in the range from 1/600 to 1/20,000 (weight/weight); or
wherein the percentage of carboxypeptidase B relative to proinsulin is in the range from 1/600 to 1/15,000 (weight/weight).

33. The method of claim 27, wherein the pH in the enzymatic cleavage is from 6.5 to 9.0; or
wherein the pH in the enzymatic cleavage is from 7.0 to 8.5.

34. The method of claim 27, wherein the temperature in the enzymatic cleavage is from 4.0° C. to 25.0° C.

35. The method of claim 27, wherein the enzymatic cleavage is performed for 4.0 hours to 55 hours.

36. The method of claim 27, wherein the proinsulin or insulin is in an analog type.

37. The method of claim 27, further comprising purifying insulin by subjecting a sample comprising the insulin converted from proinsulin to chromatography.

38. The method of claim 37, wherein the chromatography is cation exchange chromatography or reversed phase chromatography.

39. The method of claim 38, further comprising performing reversed phase chromatography or anion exchange chromatography.

40. The method of claim 27, wherein the proinsulin is partially purified by a cation exchange column or a reversed column.

41. The method of claim 27, wherein the sample containing insulin prepared by the method comprises Des-Thr(B30)-insulin impurities in the amount of less than 5%.

42. The method of claim 27, wherein the enzymatic cleavage is conducted in a Tris-HCl buffer ranging from 1 mM to 100 mM.

43. The method of claim 27, wherein a buffer in the enzymatic cleavage contains no metal ion.

44. A method for purifying insulin comprising:
(a) preparing an insulin-containing sample by the method of claim 27; and
(b) applying the sample to a chromatography process.

45. The method of claim 44, wherein the chromatography is cation exchange chromatography or reversed phase chromatography.

46. The method of claim 45, further subjecting the sample to reversed phase chromatography or anion exchange chromatography.


SEQUENCE LISTING (52 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 21 aa, Artificial Sequence
  Insulin analog, formula 1
  MISC_FEATURE (1): Xaa is glycine or alanine
  MISC_FEATURE (2): Xaa is isoleucine or alanine
  MISC_FEATURE (14): Xaa is tyrosine, glutamic acid, asparagine, histidine, lysine, alanine, or aspartic acid
  MISC_FEATURE (19): Xaa is tyrosine, glutamic acid, serine, threonine or alanine
XXVEQCCTSICSLXQLENXCN

SEQ ID NO: 4 - protein, 30 aa, Artificial Sequence
  Insulin analog, formula 2
  MISC_FEATURE (8): Xaa is glycine or alanine
  MISC_FEATURE (16): Xaa is Tyrosine, glutamic acid, serine, threonine or aspartic acid
  MISC_FEATURE (23): Xaa is glycine or alanine
  MISC_FEATURE (24): Xaa is phenylalanine or alanine
  MISC_FEATURE (25): Xaa is phenylalanine, aspartic acid, glutamic acid alanine or deletion
FVNQHLCXSHLVEALXLVCGERXXXYTPKT

SEQ ID NO: 5 - DNA, 258 bp, Artificial Sequence
  Analog 1
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtgcgattgtggaacaatgctgtaccagcatctgctccctctaccagctggagaactactgcaac

SEQ ID NO: 6 - protein, 86 aa, Artificial Sequence
  Analog 1
FVNQHLCGSHLVEALYLVCGERGFFYTPKTRREAEDLQVGQVELGGGPGAGSLQPLALEGSLQKRAIVEQCCTSICSLYQLENYCN

SEQ ID NO: 7 - DNA, 258 bp, Artificial Sequence
  Analog 2
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcgcggtggaacaatgctgtaccagcatctgctccctctaccagctggagaactactgcaac

SEQ ID NO: 8 - protein, 86 aa, Artificial Sequence
  Analog 2
FVNQHLCGSHLVEALYLVCGERGFFYTPKTRREAEDLQVGQVELGGGPGAGSLQPLALEGSLQKRGAVEQCCTSICSLYQLENYCN

SEQ ID NO: 9 - DNA, 258 bp, Artificial Sequence
  Analog 3
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaacgcgtgcaac

SEQ ID NO: 10 - protein, 86 aa, Artificial Sequence
  Analog 3
FVNQHLCGSHLVEALYLVCGERGFFYTPKTRREAEDLQVGQVELGGGPGAGSLQPLALEGSLQKRGIVEQCCTSICSLYQLENACN

SEQ ID NO: 11 - DNA, 258 bp, Artificial Sequence
  Analog 4
ttcgttaaccaacacttgtgtgcgtcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 12 - protein, 86 aa, Artificial Sequence
  Analog 4
FVNQHLCASHLVEALYLVCGERGFFYTPKTRREAEDLQVGQVELGGGPGAGSLQPLALEGSLQKRGIVEQCCTSICSLYQLENYCN

SEQ ID NO: 13 - DNA, 258 bp, Artificial Sequence
  Analog 5
ttcgttaaccaacacttgtgtggctcacacctggtggaagctctctacctagtgtgcggggaacgagcgttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 14 - protein, 86 aa, Artificial Sequence
  Analog 5
FVNQHLCGSHLVEALYLVCGERAFFYTPKTRREAEDLQVGQVELGGGPGAGSLQPLALEGSLQKRGIVEQCCTSICSLYQLENYCN

SEQ ID NO: 15 - DNA, 258 bp, Artificial Sequence
  Analog 6
ttcgttaaccaacacttgtgtggctcacacctggtggaagctctctacctagtgtgcggggaacgaggcgcgttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 16 - protein, 86 aa, Artificial Sequence
  Analog 6
FVNQHLCGSHLVEALYLVCGERGAFYTPKTRREAEDLQVGQVELGGGPGAGSLQPLALEGSLQKRGIVEQCCTSICSLYQLENYCN

SEQ ID NO: 17 - DNA, 258 bp, Artificial Sequence
  Analog 7
ttcgttaaccaacacttgtgtggctcacacctggtggaagctctctacctagtgtgcggggaacgaggcttcgcgtacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 18 - protein, 86 aa, Artificial Sequence
  Analog 7
FVNQHLCGSHLVEALYLVCGERGFAYTPKTRREAEDLQVGQVELGGGPGAGSLQPLALEGSLQKRGIVEQCCTSICSLYQLENYCN

SEQ ID NO: 19 - DNA, 261 bp, Artificial Sequence
  Analog 8
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcgaacagctggagaactactgcaactga

SEQ ID NO: 20 - protein, 86 aa, Artificial Sequence
  Analog 8
FVNQHLCGSHLVEALYLVCGERGFFYTPKTRREAEDLQVGQVELGGGPGAGSLQPLALEGSLQKRGIVEQCCTSICSLEQLENYCN

SEQ ID NO: 21 - DNA, 261 bp, Artificial Sequence
  Analog 9
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcaaccagctggagaactactgcaactga

SEQ ID NO: 22 - protein, 86 aa, Artificial Sequence
  Analog 9
FVNQHLCGSHLVEALYLVCGERGFFYTPKTRREAEDLQVGQVELGGGPGAGSLQPLALEGSLQKRGIVEQCCTSICSLNQLENYCN

SEQ ID NO: 23 - DNA, 258 bp, Artificial Sequence
  Analog 10
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctccatcagctggagaactactgcaac

SEQ ID NO: 24 - protein, 86 aa, Artificial Sequence
  Analog 10
FVNQHLCGSHLVEALYLVCGERGFFYTPKTRREAEDLQVGQVELGGGPGAGSLQPLALEGSLQKRGIVEQCCTSICSLHQLENYCN

SEQ ID NO: 25 - DNA, 258 bp, Artificial Sequence
  Analog 11
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcaagcagctggagaactactgcaac

SEQ ID NO: 26 - protein, 86 aa, Artificial Sequence
  Analog 11
FVNQHLCGSHLVEALYLVCGERGFFYTPKTRREAEDLQVGQVELGGGPGAGSLQPLALEGSLQKRGIVEQCCTSICSLKQLENYCN

SEQ ID NO: 27 - DNA, 258 bp, Artificial Sequence
  Analog 12
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaacgagtgcaac

SEQ ID NO: 28 - protein, 86 aa, Artificial Sequence
  Analog 12
FVNQHLCGSHLVEALYLVCGERGFFYTPKTRREAEDLQVGQVELGGGPGAGSLQPLALEGSLQKRGIVEQCCTSICSLYQLENECN

SEQ ID NO: 29 - DNA, 258 bp, Artificial Sequence
  Analog 13
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactcctgcaac

SEQ ID NO: 30 - protein, 86 aa, Artificial Sequence
  Analog 13
FVNQHLCGSHLVEALYLVCGERGFFYTPKTRREAEDLQVGQVELGGGPGAGSLQPLALEGSLQKRGIVEQCCTSICSLYQLENSCN

SEQ ID NO: 31 - DNA, 258 bp, Artificial Sequence
  Analog 14
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaacacctgcaac

SEQ ID NO: 32 - protein, 86 aa, Artificial Sequence
  Analog 14
FVNQHLCGSHLVEALYLVCGERGFFYTPKTRREAEDLQVGQVELGGGPGAGSLQPLALEGSLQKRGIVEQCCTSICSLYQLENTCN

SEQ ID NO: 33 - DNA, 258 bp, Artificial Sequence
  Analog 15
ttcgttaaccaacacttgtgtggctcacacctggtggaagctctcgagctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 34 - protein, 86 aa, Artificial Sequence
  Analog 15
FVNQHLCGSHLVEALELVCGERGFFYTPKTRREAEDLQVGQVELGGGPGAGSLQPLALEGSLQKRGIVEQCCTSICSLYQLENYCN

SEQ ID NO: 35 - DNA, 258 bp, Artificial Sequence
  Analog 16
ttcgttaaccaacacttgtgtggctcacacctggtggaagctctctccctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 36 - protein, 86 aa, Artificial Sequence
  Analog 16
FVNQHLCGSHLVEALSLVCGERGFFYTPKTRREAEDLQVGQVELGGGPGAGSLQPLALEGSLQKRGIVEQCCTSICSLYQLENYCN

SEQ ID NO: 37 - DNA, 258 bp, Artificial Sequence
  Analog 17
ttcgttaaccaacacttgtgtggctcacacctggtggaagctctcaccctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 38 - protein, 86 aa, Artificial Sequence
  Analog 17
FVNQHLCGSHLVEALTLVCGERGFFYTPKTRREAEDLQVGQVELGGGPGAGSLQPLALEGSLQKRGIVEQCCTSICSLYQLENYCN

SEQ ID NO: 39 - DNA, 258 bp, Artificial Sequence
  Analog 18
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcgcccagctggagaactactgcaac

SEQ ID NO: 40 - protein, 86 aa, Artificial Sequence
  Analog 18
FVNQHLCGSHLVEALYLVCGERGFFYTPKTRREAEDLQVGQVELGGGPGAGSLQPLALEGSLQKRGIVEQCCTSICSLAQLENYCN

SEQ ID NO: 41 - DNA, 258 bp, Artificial Sequence
  Analog 19
ttcgttaaccaacacttgtgtggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcgaccagctggagaactactgcaac

SEQ ID NO: 42 - protein, 86 aa, Artificial Sequence
  Analog 19
FVNQHLCGSHLVEALYLVCGERGFFYTPKTRREAEDLQVGQVELGGGPGAGSLQPLALEGSLQKRGIVEQCCTSICSLDQLENYCN

SEQ ID NO: 43 - DNA, 258 bp, Artificial Sequence
  Analog 20
ttcgttaaccaacacttgtgtggctcacacctggtggaagctctcgacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 44 - protein, 86 aa, Artificial Sequence
  Analog 20
FVNQHLCGSHLVEALDLVCGERGFFYTPKTRREAEDLQVGQVELGGGPGAGSLQPLALEGSLQKRGIVEQCCTSICSLYQLENYCN

SEQ ID NO: 45 - DNA, 258 bp, Artificial Sequence
  Analog 21
ttcgttaaccaacacttgtgtggctcacacctggtggaagctctctacctagtgtgcggggaacgaggcttcgactacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 46 - protein, 86 aa, Artificial Sequence
  Analog 21
FVNQHLCGSHLVEALYLVCGERGFDYTPKTRREAEDLQVGQVELGGGPGAGSLQPLALEGSLQKRGIVEQCCTSICSLYQLENYCN

SEQ ID NO: 47 - DNA, 258 bp, Artificial Sequence
  Analog 22
ttcgttaaccaacacttgtgtggctcacacctggtggaagctctctacctagtgtgcggggaacgaggcttcgagtacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 48 - protein, 86 aa, Artificial Sequence
  Analog 22
FVNQHLCGSHLVEALYLVCGERGFEYTPKTRREAEDLQVGQVELGGGPGAGSLQPLALEGSLQKRGIVEQCCTSICSLYQLENYCN

SEQ ID NO: 49 - DNA, 255 bp, Artificial Sequence
  Analog 23
ttcgttaaccaacacttgtgtggctcacacctggtggaagctctctacctagtgtgcggggaacgaggcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcgaacagctggagaactactgcaac

SEQ ID NO: 50 - protein, 85 aa, Artificial Sequence
  Analog 23
FVNQHLCGSHLVEALYLVCGERGFYTPKTRREAEDLQVGQVELGGGPGAGSLQPLALEGSLQKRGIVEQCCTSICSLEQLENYCN

SEQ ID NO: 51 - DNA, 255 bp, Artificial Sequence
  Analog 24
ttcgttaaccaacacttgtgtggctcacacctggtggaagctctcgagctagtgtgcggggaacgaggcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctcgcccagctggagaactactgcaac

SEQ ID NO: 52 - protein, 85 aa, Artificial Sequence
  Analog 24
FVNQHLCGSHLVEALELVCGERGFYTPKTRREAEDLQVGQVELGGGPGAGSLQPLALEGSLQKRGIVEQCCTSICSLAQLENYCN